(19) United States
(12) Patent Application Publication
(10) Pub. No.: US 2006/0084675 A1
(43) Pub. Date: Apr. 20, 2006

(54) Combined treatment with artesunate and an epidermal growth factor receptor kinase inhibitor

(76) Inventors: Thomas Efferth, Heidelberg (DE); Marc-Eric Halatsch, Heidelberg (DE)

Correspondence Address: OSI PHARMACEUTICALS, INC., 58 SOUTH SERVICE ROAD, MELVILLE, NY, 11747, US

(21) Appl. No.: 11/252,304

(22) Filed: Oct. 17, 2005

Related U.S. Application Data
(60) Provisional application No. 60/619,829, filed on Oct. 18, 2004.

(51) Int. Cl. A61K 31/47

(52) U.S. Cl. 514/310

(57) ABSTRACT

The present invention provides a method for treating tumors or tumor metastases in a patient, comprising administering to the patient simultaneously or sequentially a therapeutically effective amount of an EGFR kinase inhibitor and artesunate combination, with or without additional agents or treatments, such as other anti-cancer drugs or radiation therapy. The invention also encompasses a pharmaceutical composition that is comprised of an EGFR kinase inhibitor and artesunate combination in combination with a pharmaceutically acceptable carrier. A preferred example of an EGFR kinase inhibitor that can be used in practicing this invention is the compound erlotinib HCl (also known as Tarceva™).

### CROSS-REFERENCE TO RELATED APPLICATIONS

This application claims the benefit of U.S. Provisional Application No. 60/619,829, filed Oct. 18, 2004, which is herein incorporated by reference in its entirety.

### BACKGROUND OF THE INVENTION

The present invention is directed to compositions and methods for treating cancer patients. In particular, the present invention is directed to combined treatment of patients with artesunate and an epidermal growth factor receptor (EGFR) kinase inhibitor.

Cancer is a generic name for a wide range of cellular malignancies characterized by unregulated growth, lack of differentiation, and the ability to invade local tissues and metastasize. These neoplastic malignancies affect, with various degrees of prevalence, every tissue and organ in the body.

A multitude of therapeutic agents have been developed over the past few decades for the treatment of various types of cancer. The most commonly used types of anticancer agents include: DNA-alkylating agents (e.g., cyclophosphamide, ifosfamide), antimetabolites (e.g., methotrexate, a folate antagonist, and 5-fluorouracil, a pyrimidine antagonist), microtubule disrupters (e.g., vincristine, vinblastine, paclitaxel), DNA intercalators (e.g., doxorubicin, daunomycin, cisplatin), and hormone therapy (e.g., tamoxifen, flutamide).

Glioblastoma multiforme (GBM) is the most aggressive form of the primary brain tumors known collectively as gliomas. These tumors arise from glial cells in the brain during childhood and in adults. They only rarely metastasize, but cause symptoms by mass effects and invasion of other areas of the brain. Treatment for GBM can include surgery to remove the maximum volume of tumor, radiation therapy, and chemotherapy with agents such as nitrosoureas (e.g. BCNU, CCNU), PVC (procarbazine, CCNU and vincristine), temozolomide, thalidomide, and tamoxifen. Additionally, new agents such as the EGFR kinase inhibitor erlotinib ([6,7-bis(2-methoxyethoxy)-4-quinazolin-4-yl]-(3-ethynylphenyl)amine, e.g. erlotinib HCl, also known as Tarceva™ or OSI-774) have shown promise in treatment (Prados, M. et. al. (2003) Proc. Am. Soc. Clin. Oncol. 22:99, (abstr. 394)). However, the prognosis for GBM remains poor, with the mean survival period being only 12-14 months from diagnosis.

Over-expression of the epidermal growth factor receptor (EGFR) kinase, or its ligand TGF-alpha, is frequently associated with many cancers, including breast, lung, colorectal, brain (e.g. gliomas), and head and neck cancers (Salomon, D. S., et al. (1995) Crit. Rev. Oncol. Hematol. 19:183-232; Wells, A. (2000) Signal, 1:4-11), and is believed to contribute to the malignant growth of these tumors. A specific deletion-mutation in the EGFR gene has also been found to increase cellular tumorigenicity (Halatsch, M-E. et al. (2000) J. Neurosurg. 92:297-305; Archer, G. E., et al. (1999) Clin. Cancer Res. 5:2646-2652). Activation of EGFR stimulated signaling pathways promote multiple processes that are potentially cancer-promoting, e.g. proliferation, angiogenesis, cell motility and invasion, decreased apoptosis and induction of drug resistance. The development for use as anti-tumor agents of compounds that directly inhibit the kinase activity of the EGFR, as well as antibodies that reduce EGFR kinase activity by blocking EGFR activation, are areas of intense research effort (de Bono J. S. and Rowinsky, E. K. (2002) Trends in Mol. Medicine 8:S19-S26; Dancey, J. and Sausville, E. A. (2003) Nature Rev. Drug Discovery 2:92-313). Several studies have demonstrated or disclosed that some EGFR kinase inhibitors can improve tumor cell or neoplasia killing when used in combination with certain other anti-cancer or chemotherapeutic agents or treatments (e.g. Raben, D. et al. (2002) Semin. Oncol. 29:3746; Herbst, R. S. et al. (2001) Expert Opin. Biol. Ther. 1:719-732; Magne, N et al. (2003) Clin. Can. Res. 9:4735-4732; Magne, N. et al. (2002) British Journal of Cancer 86:819-827; Torrance, C. J. et al. (2000) Nature Med. 6:1024-1028; Gupta, R. A. and DuBois, R. N. (2000) Nature Med. 6:974-975; Tortora, et al. (2003) Clin. Cancer Res. 9:1566-1572; Solomon, B. et al (2003) Int. J. Radiat. Oncol. Biol. Phys. 55:713-723; Krishnan, S. et al. (2003) Frontiers in Bioscience 8, e1-13; Huang, S et al. (1999) Cancer Res. 59:1935-1940; Contessa, J. N. et al. (1999) Clin. Cancer Res. 5:405-411; Li, M. et al. Clin. (2002) Cancer Res. 8:3570-3578; Ciardiello, F. et al. (2003) Clin. Cancer Res. 9:1546-1556; Ciardiello, F. et al. (2000) Clin. Cancer Res. 6:3739-3747; Grunwald, V. and Hidalgo, M. (2003) J. Nat. Cancer Inst. 95:851-867; Seymour L. (2003) Current Opin. Investig. Drugs 4(6):658-666; Khalil, M. Y. et al. (2003) Expert Rev. Anticancer Ther.3:367-380; Bulgaru, A. M. et al. (2003) Expert Rev. Anticancer Ther.3:269-279; Dancey, J. and Sausville, E. A. (2003) Nature Rev. Drug Discovery 2:92-313; Kim, E. S. et al. (2001) Current Opinion Oncol. 13:506-513; Arteaga, C. L. and Johnson, D. H. (2001) Current Opinion Oncol. 13:491-498; Ciardiello, F. et al. (2000) Clin. Cancer Res. 6:2053-2063; Patent Publication Nos: U.S. 2003/0108545; U.S. 2002/0076408; and U.S. 2003/0157104; and International Patent Publication Nos: WO 99/60023; WO 01/12227; WO 02/055106; WO 03/088971; WO 01/34574; WO 01/76586; WO 02/05791; and WO 02/089842).

OSI-774 (also known as erlotinib HCl or Tarceva™) is a quinazoline that inhibits the tyrosine kinase activity of EGFR and induces apoptosis and cell cycle arrest (Moyer, J. D., et al. (1997) Cancer Res. 57:4838-4848; Norman P. (2001) Curr. Opin. Investig. Drugs 2:298-304). In a recent study, it has been shown that the ability of GBM cell lines to induce EGFR mRNA expression after exposure to OSI-774 was associated with decreased susceptibility to the anti-tumorigenic effect of OSI-774 as a cellular effort to counteract functional EGFR inhibition by this drug (Halatsch et al. (2004) J. Neurosurg. 100:523-533).

Another novel compound with profound cytotoxicity against tumor cells is artesunate (ART). This is a semisynthetic derivative of artemisinin, the active principle of Artemisia annua L. Though initially described as an anti-malarial drug, ART proved to be active in 55 cell lines of the National Cancer Institute (N.C.I.), U.S.A. (Efferth, T. (2001) Int. J. Oncol. 18:767-773). Mining the National Cancer Institute's database for the mRNA expression microarray data for 465 genes showed that EGFR gene expression correlated inversely with the cellular sensitivity to ART, indicating that EGFR is a determinant of cellular response to ART (Efferth, T. (2003) Mol. Pharmacol. 64:382-394).

An anti-neoplastic drug would ideally kill cancer cells selectively, with a wide therapeutic index relative to its toxicity towards non-malignant cells. It would also retain its efficacy against malignant cells, even after prolonged exposure to the drug. Unfortunately, none of the current chemotherapies possess such an ideal profile. Instead, most possess very narrow therapeutic indexes. Furthermore, cancerous cells exposed to slightly sub-lethal concentrations of a chemotherapeutic agent will very often develop resistance to such an agent, and quite often cross-resistance to several other antineoplastic agents as well.

Thus, there is a need for more efficacious treatment for neoplasia and other proliferative disorders. Strategies for enhancing the therapeutic efficacy of existing drugs have involved changes in the schedule for their administration, and also their use in combination with other anticancer or biochemical modulating agents. Combination therapy is well known as a method that can result in greater efficacy and diminished side effects relative to the use of the therapeutically relevant dose of each agent alone. In some cases, the efficacy of the drug combination is additive (the efficacy of the combination is approximately equal to the sum of the effects of each drug alone), but in other cases the effect is synergistic (the efficacy of the combination is greater than the sum of the effects of each drug given alone).

However, there remains a critical need for improved treatments for gliomas and other cancers. This invention provides anti-cancer combination therapies that reduce the dosages for individual components required for efficacy, thereby decreasing side effects associated with each agent, while maintaining or increasing therapeutic value. The invention described herein provides new drug combinations, and methods for using drug combinations in the treatment of gliomas and other cancers.

### SUMMARY OF THE INVENTION

The present invention provides a method for treating tumors or tumor metastases in a patient, comprising administering to the patient simultaneously or sequentially a therapeutically effective amount of an EGFR kinase inhibitor and artesunate combination, with or without additional agents or treatments, such as other anti-cancer drugs or radiation therapy.

The invention also encompasses a pharmaceutical composition that is comprised of an EGFR kinase inhibitor and artesunate combination in combination with a pharmaceutically acceptable carrier.

A preferred example of an EGFR kinase inhibitor that can be used in practicing this invention is the compound erlotinib HCl (also known as Tarceva™).

### BRIEF DESCRIPTION OF THE FIGURES

FIG. 1: Treatment responses of GBM cell lines with OSI-774 assessed by the growth inhibition assay. (a) Dose-response curves of U-87MG and U-87MG.ΔEGFR cells after exposure to 0.1 to 10 μM OSI-774. (b) Treatment of transduced and non-transduced GBM cell lines with 1 μM OSI-774. Each data point or bar represents mean±SD of four measurements.

FIG. 2: Combination treatment of five EGFR-transduced GBM cell lines with ART and OSI-774 assessed by the growth inhibition assay. Cell growth inhibition by ART alone (open symbols) or by ART plus OSI-774 (1 μM) (closed symbols) is expressed as percentage of controls and is plotted against μM ART for U-87MG.ΔEGFR, cells transduced with deletion-mutant EGFR; U-87MG.DK-2N, cells transduced cells with tyrosine kinase deficient EGFR; U-87MG.WT-2N, cells transduced with wild-type EGFR; U-87MG.LUX, cells transduced with expression vector only; and U-87MG, non-transduced parental cells. Data represent mean±SD of four measurements.

FIG. 3: Combination treatment of nine non-transduced glioblastoma cell lines with OSI-774 and ART assessed by the growth inhibition assay. Cell growth inhibition by ART alone (open symbols) or by ART plus OSI-774 (1 μM) (closed symbols) is expressed as percentage of controls.

FIG. 4: Isobologram analysis (50% isodose) of OSI-774 and ART combination treatments. Growth inhibition expressed as % of control after exposure to serial dilutions of both drugs is plotted against (a) OSI-774 or (b) ART. The isobolographic plot (c) shows the IC₅₀ values of combined treatments. The solid bold diagonal line represents additivity. Data points that fall to the left of this line indicate supra-additivity or synergy. Thus, synergy is shown. Data represent mean±SD of four measurements.

FIG. 5: Representative examples of genomic consensus imbalances obtained by comparative genomic hybridization that correlated with IC₅₀ values for ART and OSI-774.

FIG. 6: Dendrograms and clustered image map obtained by hierarchical cluster analysis (complete linkage method). The dendrogram to the right shows the clustering of genomic imbalances (gains or losses) detected by comparative genomic hybridization (see FIG. 7) and the top dendrogram of eight GBM cell lines. Light fields indicate ‘imbalance not present’, and dark fields indicate ‘imbalance present’.

FIG. 7: Inhibition concentrations 50% (IC₅₀) of GBM cell lines after treatment with ART and OSI-774 in growth inhibition assays.

FIG. 8: Statistical analysis of genomic imbalances in GBM cell lines subjected to hierarchical cluster analysis in FIG. 5.

FIG. 9: Genes of genomic imbalances that correlate with IC₅₀ values for ART and

### DETAILED DESCRIPTION OF THE INVENTION

The term “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient. The term “treatment” as used herein, unless otherwise indicated, refers to the act of treating.

The phrase “a method of treating” or its equivalent, when applied to, for example, cancer refers to a procedure or course of action that is designed to reduce or eliminate the number of cancer cells in an animal, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an animal, is nevertheless deemed an overall beneficial course of action.

The term “therapeutically effective agent” means a composition that will elicit the biological or medical response of a tissue, system, animal or human that is being sought by the researcher, veterinarian, medical doctor or other clinician.

The term “therapeutically effective amount” or “effective amount” means the amount of the subject compound or combination that will elicit the biological or medical response of a tissue, system, animal or human that is being sought by the researcher, veterinarian, medical doctor or other clinician.

The data presented in the Examples herein below demonstrate that co-administration of artesunate with an EGFR kinase inhibitor is effective for treatment of patients with advanced cancers, such as gliomas, e.g. glioblastoma multiforme. Accordingly, the present invention provides a method for treating tumors or tumor metastases in a patient, comprising administering to the patient simultaneously or sequentially a therapeutically effective amount of an EGFR kinase inhibitor and artesunate combination. In this method, the cancer present in the patient can be any of those referred to herein below, including glioma, and glioblastoma multiforme.

In any of the methods of the present invention, the administration of agents simultaneously can be performed by separately administering agents at the same time, or together as a fixed combination. Also, in any of the methods of the present invention, the administration of agents sequentially can be in any order.

The present invention further provides a method for treating tumors or tumor metastases in a patient, comprising administering to the patient simultaneously or sequentially a therapeutically effective amount of an EGFR kinase inhibitor and artesunate combination, and in addition, one or more other cytotoxic, chemotherapeutic or anti-cancer agents, or compounds that enhance the effects of such agents.

In the context of this invention, additional other cytotoxic, chemotherapeutic or anti-cancer agents, or compounds that enhance the effects of such agents, include, for example: alkylating agents or agents with an alkylating action, such as cyclophosphamide (CTX; e.g. cytoxan®), chlorambucil (CHL; e.g. leukeran®), cisplatin (CisP; e.g. platinol®) busulfan (e.g. myleran®), melphalan, carmustine (BCNU), streptozotocin, triethylenemelamine (TEM), mitomycin C, and the like; anti-metabolites, such as methotrexate (MTX), etoposide (VP16; e.g. vepesid®), 6-mercaptopurine (6 MP), 6-thiocguanine (6TG), cytarabine (Ara-C), 5-fluorouracil (5-FU), capecitabine (e.g. Xeloda®), dacarbazine (DTIC), and the like; antibiotics, such as actinomycin D, doxorubicin (DXR; e.g. adriamycin®), daunorubicin (daunomycin), bleomycin, mithramycin and the like; alkaloids, such as vinca alkaloids such as vincristine (VCR), vinblastine, and the like; and other antitumor agents, such as paclitaxel (e.g. taxol®) and pactitaxel derivatives, the cytostatic agents, glucocorticoids such as dexamethasone (DEX; e.g. decadron®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also beused as additional agents: amifostine (e.g. ethyol®), dactinomycin, mechlorethamine (nitrogen mustard), streptozocin, cyclophosphamide, lomustine (CCNU), doxorubicin lipo (e.g. doxil®), gemcitabine (e.g. gemzar®), daunorubicin lipo (e.g. daunoxome®), procarbazine, mitomycin, docetaxel (e.g. taxotere®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon alpha, interferon beta, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, and chlorambucil.

The present invention further provides a method for treating tumors or tumor metastases in a patient, comprising administering to said patient simultaneously or sequentially a therapeutically effective amount of an EGFR kinase inhibitor and artesunate combination, and in addition, one or more anti-hormonal agents. As used herein, the term “anti-hormonal agent” includes natural or synthetic organic or peptidic compounds that act to regulate or inhibit hormone action on tumors.

Antihormonal agents include, for example: steroid receptor antagonists, anti-estrogens such as tamoxifen, raloxifene, aromatase inhibiting 4(5)-imidazoles, other aromatase inhibitors, 42-hydroxytamoxifen, trioxifene, keoxifene, LY 117018, onapristone, and toremifene (e.g. Fareston®); anti-androgens such as flutamide, nilutamide, bicalutamide, leuprolide, and goserelin; and pharmaceutically acceptable salts, acids or derivatives of any of the above; agonists and/or antagonists of glycoprotein hormones such as follicle stimulating hormone (FSH), thyroid stimulating hormone (TSH), and luteinizing hormone (LH) and LHRH (leuteinizing hormone-releasing hormone); the LHRH agonist goserelin acetate, commercially available as Zoladex® (AstraZeneca); the LHRH antagonist D-alaninamide N-acetyl-3-(2-naphthalenyl)-D-alanyl-4-chloro-D-phenylalanyl-3-(3-pyridinyl)-D-alanyl-L-seryl-N-6-(3-pyridinylcarbonyl)-L-lysyl-N-6-(3-pyridinylcarbonyl)-D-lysyl-L-leucyl-N-6-(1-methylethyl)-L-lysyl-L-proline (e.g Antide®, Ares-Serono); the LHRH antagonist ganirelix acetate; the steroidal anti-androgens cyproterone acetate (CPA) and megestrol acetate, commercially available as Megace® (Bristol-Myers Oncology); the nonsteroidal anti-androgen flutamide (2-methyl-N-[4,20-nitro-3-(trifluoromethyl) phenylpropanamide), commercially available as Eulexin® (Schering Corp.); the non-steroidal anti-androgen nilutamide, (5,5-dimethyl-3-[4-nitro-3-(trifluoromethyl-4-nitrophenyl)-4,4-dimethyl-imidazolidine-dione); and antagonists for other non-permissive receptors, such as antagonists for RAR, RXR, TR, VDR, and the like.

The use of the cytotoxic and other anticancer agents described above in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

The present invention further provides a method for treating tumors or tumor metastases in a patient, comprising administering to said patient simultaneously or sequentially a therapeutically effective amount of an EGFR kinase inhibitor and artesunate combination, and in addition one or more angiogenesis inhibitors.

Anti-angiogenic agents include, for example: VEGFR inhibitors, such as SU-5416 and SU-6668 (Sugen Inc. of South San Francisco, Calif., USA), or as described in, for example International Application Nos. WO 99/24440, WO 99/62890, WO 95/21613, WO 99/61422, WO 98/50356, WO 99/10349, WO 97/32856, WO 97/22596, WO 98/54093, WO 98/02438, WO 99/16755, and WO 98/02437, and U.S. Pat. Nos. 5,883,113, 5,886,020, 5,792,783, 5,834,504 and 6,235,764; VEGF inhibitors such as IM862 (Cytran Inc. of Kirkland, Wash., USA); angiozyme, a synthetic ribozyme from Ribozyme (Boulder, Colo.) and Chiron (Emeryville, Calif.); and antibodies to VEGF, such as bevacizumab (e.g. Avastin™, Genentech, South San Francisco, Calif.), a recombinant humanized antibody to VEGF; integrin receptor antagonists and integrin antagonists, such as to αᵥβ₃, αᵥβ₅ and αᵥβ₆ integrins, and subtypes thereof, e.g. cilengitide (EMD 121974), or the anti-integrin antibodies, such as for example αᵥβ₃ specific humanized antibodies (e.g. Vitaxin®); factors such as IFN-alpha (U.S. Pat. Nos. 41530901, 4,503,035, and 5,231,176); angiostatin and plasminogen fragments (e.g. kringle 1-4, kringle 5, kringle 1-3 (O'Reilly, M. S. et al. (1994) Cell 79:315-328; Cao et al. (1996) J. Biol. Chem. 271:29461-29467; Cao et al. (1997) J. Biol. Chem. 272:22924-22928); endostatin (O'Reilly, M. S. et al. (1997) Cell 88:277; and International Patent Publication No. WO 97/15666); thrombospondin (TSP-1; Frazier, (1991) Curr. Opin. Cell Biol. 3:792); platelet factor 4 (PF4); plasminogen activator/urokinase inhibitors; urokinase receptor antagonists; heparinases; fumagillin analogs such as TNP-4701; suramin and suramin analogs; angiostatic steroids; bFGF antagonists; flk-1 and flt-1 antagonists; anti-angiogenesis agents such as MMP-2 (matrix-metalloproteinase 2) inhibitors and MMP-9 (matrix-metalloproteinase 9) inhibitors. Examples of useful matrix metalloproteinase inhibitors are described in International Patent Publication Nos. WO 96/33172, WO 96/27583, WO 98/07697, WO 98/03516, WO 98/34918, WO 98/34915, WO 98/33768, WO 98/30566, WO 90/05719, WO 99/52910, WO 99/52889, WO 99/29667, and WO 99/07675, European Patent Publication Nos. 818,442, 780,386, 1,004,578, 606,046, and 931,788; Great Britain Patent Publication No. 9912961, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

The present invention further provides a method for treating tumors or tumor metastases in a patient, comprising administering to the patient simultaneously or sequentially a therapeutically effective amount of an EGFR kinase inhibitor and artesunate combination, and in addition one or more tumor cell pro-apoptotic or apoptosis-stimulating agents.

The present invention further provides a method for treating tumors or tumor metastases in a patient, comprising administering to said patient simultaneously or sequentially a therapeutically effective amount of an EGFR kinase inhibitor and artesunate combination, and in addition one or more signal transduction inhibitors.

Signal transduction inhibitors include, for example: erbB2 receptor inhibitors, such as organic molecules, or antibodies that bind to the erbB2 receptor, for example, trastuzumab (e.g. Herceptin®); inhibitors of other protein tyrosine-kinases, e.g. imitinib (e.g. Gleevec®); ras inhibitors; raf inhibitors; MEK inhibitors; mTOR inhibitors; cyclin dependent kinase inhibitors; protein kinase C inhibitors; and PDK-1 inhibitors (see Dancey, J. and Sausville, E. A. (2003) Nature Rev. Drug Discovery 2:92-313, for a description of several examples of such inhibitors, and their use in clinical trials for the treatment of cancer).

ErbB2 receptor inhibitors include, for example: ErbB2 receptor inhibitors, such as GW-282974 (Glaxo Wellcome plc), monoclonal antibodies such as AR-209 (Aronex Pharmaceuticals Inc. of The Woodlands, Tex., USA) and 2B-1 (Chiron), and erbB2 inhibitors such as those described in International Publication Nos. WO 98/02434, WO 99/35146, WO 99/35132, WO 98/02437, WO 97/13760, and WO 95/19970, and U.S. Pat. Nos. 5,587,458, 5,877,305, 6,465,449 and 6,541,481.

The present invention further thus provides a method for treating tumors or tumor metastases in a patient, comprising administering to said patient simultaneously or sequentially a therapeutically effective amount of an EGFR kinase inhibitor and artesunate combination, and in addition an anti-HER2 antibody or an immunotherapeutically active fragment thereof.

The present invention further provides a method for treating tumors or tumor metastases in a patient, comprising administering to said patient simultaneously or sequentially a therapeutically effective amount of an EGFR kinase inhibitor and artesunate combination, and in addition one or more additional anti-proliferative agents.

Additional antiproliferative agents include, for example: Inhibitors of the enzyme farnesyl protein transferase and inhibitors of the receptor tyrosine kinase PDGFR, including the compounds disclosed and claimed in U.S. Pat. Nos. 6,080,769, 6,194,438, 6,258,824, 6,586,447, 6,071,935, 6,495,564, 6,150,377, 6,596,735 and 6,479,513, and International Patent Publication WO 01/40217.

The present invention further provides a method for treating tumors or tumor metastases in a patient, comprising administering to the patient simultaneously or sequentially a therapeutically effective amount of an EGFR kinase inhibitor and artesunate combination, and in addition a COX II (cyclooxygenase II) inhibitor. Examples of useful COX-II inhibitors include alecoxib (e.g. Celebrex™), valdecoxib, and rofecoxib.

The present invention further provides a method for treating tumors or tumor metastases in a patient, comprising administering to the patient simultaneously or sequentially a therapeutically effective amount of an EGFR kinase inhibitor and artesunate combination, and in addition treatment with radiation or a radiopharmaceutical.

The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111. Where the EGFR kinase inhibitor according to this invention is an antibody, it is also possible to label the antibody with such radioactive isotopes.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anticancer agents. Parameters of adjuvant radiation therapies are, for example, contained in International Patent Publication WO 99/60023.

The present invention further provides a method for treating tumors or tumor metastases in a patient, comprising administering to the patient simultaneously or sequentially a therapeutically effective amount of an EGFR kinase inhibitor and artesunate combination, and in addition treatment with one or more agents capable of enhancing antitumor immune responses.

Agents capable of enhancing antitumor immune responses include, for example: CTLA4 (cytotoxic lymphocyte antigen 4) antibodies (e.g. MDX-CTLA4), and other agents capable of blocking CTLA4. Specific CTLA4 antibodies that can be used in the present invention include those described in U.S. Pat. No. 6,682,736.

The present invention further provides a method for reducing the side effects caused by the treatment of tumors or tumor metastases in a patient with an EGFR kinase inhibitor or artesunate, comprising administering to the patient simultaneously or sequentially a therapeutically effective amount of an EGFR kinase inhibitor and artesunate combination, in amounts that are effective to produce an additive, or a superadditive or synergistic antitumor effect, and that are effective at inhibiting the growth of the tumor.

The present invention further provides a method for the treatment of cancer, comprising administering to a subject in need of such treatment (i) an effective first amount of an EGFR kinase inhibitor, or a pharmaceutically acceptable salt thereof; and (ii) an effective second amount of artesunate. In this method the cancer can be any of those referred to herein below, including glioma, and glioblastoma multiforme.

The present invention also provides a method for the treatment of cancer, comprising administering to a subject in need of such treatment (i) a sub-therapeutic first amount of an EGFR kinase inhibitor, or a pharmaceutically acceptable salt thereof; and (ii) a sub-therapeutic second amount of artesunate. In this method the cancer can be any of those referred to herein below, including glioma, and glioblastoma multiforme.

The present invention also provides a method for the treatment of cancer, comprising administering to a subject in need of such treatment (i) an effective first amount of an EGFR kinase inhibitor, or a pharmaceutically acceptable salt thereof; and (ii) a sub-therapeutic second amount of artesunate. In this method the cancer can be any of those referred to herein below, including glioma, and glioblastoma multiforme.

The present invention also provides a method for the treatment of cancer, comprising administering to a subject in need of such treatment (i) a sub-therapeutic first amount of an EGFR kinase inhibitor, or a pharmaceutically acceptable salt thereof; and (ii) an effective second amount of artesunate. In this method the cancer can be any of those referred to herein below, including glioma, and glioblastoma multiforme.

In the preceding methods the order of administration of the first and second amounts can be simultaneous or sequential, i.e. artesunate can be administered before the EGFR kinase inhibitor, after the EGFR inhibitor, or at the same time as the EGFR kinase inhibitor.

In the context of this invention, an “effective amount” of an agent or therapy is as defined above. A “sub-therapeutic amount” of an agent or therapy is an amount less than the effective amount for that agent or therapy, but when combined with an effective or sub-therapeutic amount of another agent or therapy can produce a result desired by the physician, due to, for example, synergy in the resulting efficacious effects, or reduced side effects.

Additionally, the present invention provides a pharmaceutical composition comprising an EGFR inhibitor and artesunate in a pharmaceutically acceptable carrier.

As used herein, the term “patient” preferably refers to a human in need of treatment with an EGFR kinase inhibitor for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an EGFR kinase inhibitor.

In a preferred embodiment, the patient is a human in need of treatment for cancer, or a precancerous condition or lesion. The cancer is preferably any cancer treatable, either partially or completely, by administration of an EGFR kinase inhibitor. The cancer may be, for example, lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, gliomas, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions.

For purposes of the present invention, “co-administration of” and “co-administering” artesunate with an EGFR kinase inhibitor (both components referred to hereinafter as the “two active agents”) refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. Artesunate can be administered prior to, at the same time as, or subsequent to administration of the EGFR kinase inhibitor, or in some combination thereof. Where the EGFR kinase inhibitor is administered to the patient at repeated intervals, e.g., during a standard course of treatment, artesunate can be administered prior to, at the same time as, or subsequent to, each administration of the EGFR kinase inhibitor, or some combination thereof, or at different intervals in relation to the EGFR kinase inhibitor treatment, or in a single dose prior to, at any time during, or subsequent to the course of treatment with the EGFR kinase inhibitor.

The EGFR kinase inhibitor will typically be administered to the patient in a dose regimen that provides for the most effective treatment of the cancer (from both efficacy and safety perspectives) for which the patient is being treated, as known in the art, and as disclosed, e.g. in International Patent Publication No. WO 01/34574. In conducting the treatment method of the present invention, the EGFR kinase inhibitor can be administered in any effective manner known in the art, such as by oral, topical, intravenous, intra-peritoneal, intramuscular, intra-articular, subcutaneous, intranasal, intra-ocular, vaginal, rectal, or intradermal routes, depending upon the type of cancer being treated, the type of EGFR kinase inhibitor being used (e.g., small molecule, antibody, RNAi, ribozyme or antisense construct), and the medical judgement of the prescribing physician as based, for example, on the results of published clinical studies.

The amount of EGFR kinase inhibitor administered and the timing of EGFR kinase inhibitor administration will depend on the type (species, gender, age, weight, etc.) and condition of the patient being treated, the severity of the disease or condition being treated, and on the route of administration. For example, small molecule EGFR kinase inhibitors can be administered to a patient in doses ranging from 0.001 to 100 mg/kg of body weight per day or per week in single or divided doses, or by continuous infusion (see for example, International Patent Publication No. WO 01/34574). In particular, erlotinib HCl can be administered to a patient in doses ranging from 5-200 mg per day, or 100-1600 mg per week, in single or divided doses, or by continuous infusion. A preferred dose is 150 mg/day. Antibody-based EGFR kinase inhibitors, or antisense, RNAi or ribozyme constructs, can be administered to a patient in doses ranging from 0.1 to 100 mg/kg of body weight per day or per week in single or divided doses, or by continuous infusion. In some instances, dosage levels below the lower limit of the aforesaid range may be more than adequate, while in other cases still larger doses may be employed without causing any harmful side effect, provided that such larger doses are first divided into several small doses for administration throughout the day.

The EGFR kinase inhibitors and artesunate can be administered either separately or together by the same or different routes, and in a wide variety of different dosage forms. For example, the EGFR kinase inhibitor is preferably administered orally or parenterally. Artesunate is preferably administered orally or parenterally. Where the EGFR kinase inhibitor is erlotinib HCl (Tarceva™), oral administration is preferable.

The EGFR kinase inhibitor can be administered with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Administration of such dosage forms can be carried out in single or multiple doses. Carriers include solid diluents or fillers, sterile aqueous media and various non-toxic organic solvents, etc. Oral pharmaceutical compositions can be suitably sweetened and/or flavored.

The EGFR kinase inhibitor and artesunate can be combined together with various pharmaceutically acceptable inert carriers in the form of sprays, creams, salves, suppositories, jellies, gels, pastes, lotions, ointments, and the like. Administration of such dosage forms can be carried out in single or multiple doses. Carriers include solid diluents or fillers, sterile aqueous media, and various non-toxic organic solvents, etc.

All formulations comprising proteinaceous EGFR kinase inhibitors should be selected so as to avoid denaturation and/or degradation and loss of biological activity of the inhibitor.

Methods of preparing pharmaceutical compositions comprising an EGFR kinase inhibitor are known in the art, and are described, e.g. in International Patent Publication No. WO 01/34574. Methods of preparing pharmaceutical compositions comprising artesunate are also well known in the art (e.g. Singh, N. P. and Verma, K. B. (2002) Archive Oncol. 10(4):279-280). In view of the teaching of the present invention, methods of preparing pharmaceutical compositions comprising both an EGFR kinase inhibitor and artesunate will be apparent from the above-cited publications and from other known references, such as Remington's Pharmaceutical Sciences, Mack Publishing Company, Easton, Pa., 18^{th} edition (1990).

For oral administration of EGFR kinase inhibitors, tablets containing one or both of the active agents are combined with any of various excipients such as, for example, micro-crystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinyl pyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the EGFR kinase inhibitor may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration of either or both of the active agents, solutions in either sesame or peanut oil or in aqueous propylene glycol may be employed, as well as sterile aqueous solutions comprising the active agent or a corresponding water-soluble salt thereof. Such sterile aqueous solutions are preferably suitably buffered, and are also preferably rendered isotonic, e.g., with sufficient saline or glucose. These particular aqueous solutions are especially suitable for intravenous, intramuscular, subcutaneous and intraperitoneal injection purposes. The oily solutions are suitable for intra-articular, intramuscular and subcutaneous injection purposes. The preparation of all these solutions under sterile conditions is readily accomplished by standard pharmaceutical techniques well known to those skilled in the art. Any parenteral formulation selected for administration of proteinaceous EGFR kinase inhibitors should be selected so as to avoid denaturation and loss of biological activity of the inhibitor.

Additionally, it is possible to topically administer either or both of the active agents, by way of, for example, creams, lotions, jellies, gels, pastes, ointments, salves and the like, in accordance with standard pharmaceutical practice. For example, a topical formulation comprising either an EGFR kinase inhibitor or artesunate in about 0.1% (w/v) to about 5% (w/v) concentration can be prepared.

For veterinary purposes, the active agents can be administered separately or together to animals using any of the forms and by any of the routes described above. In a preferred embodiment, the EGFR kinase inhibitor is administered in the form of a capsule, bolus, tablet, liquid drench, by injection or as an implant. As an alternative, the EGFR kinase inhibitor can be administered with the animal feedstuff, and for this purpose a concentrated feed additive or premix may be prepared for a normal animal feed. The artesunate is preferably administered in the form of liquid drench, by injection or as an implant. Such formulations are prepared in a conventional manner in accordance with standard veterinary practice.

The present invention further provides a kit comprising a single container comprising both an EGFR kinase inhibitor and artesunate. The present invention further provides a kit comprising a first container comprising an EGFR kinase inhibitor and a second container comprising artesunate. In a preferred embodiment, the kit containers may further include a pharmaceutically acceptable carrier. The kit may further include a sterile diluent, which is preferably stored in a separate additional container. The kit may further include a package insert comprising printed instructions directing the use of the combined treatment as a method for treating cancer.

As used herein, the term “EGFR kinase inhibitor” refers to any EGFR kinase inhibitor that is currently known in the art or that will be identified in the future, and includes any chemical entity that, upon administration to a patient, results in inhibition of a biological activity associated with activation of the EGF receptor in the patient, including any of the downstream biological effects otherwise resulting from the binding to EGFR of its natural ligand. Such EGFR kinase inhibitors include any agent that can block EGFR activation or any of the downstream biological effects of EGFR activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the EGF receptor, thereby making the receptor inaccessible to its natural ligand so that its normal biological activity is prevented or reduced. Alternatively, such an inhibitor can act by modulating the dimerization of EGFR polypeptides, or interaction of EGFR polypeptide with other proteins, or enhance ubiquitination and endocytotic degradation of EGFR. EGFR kinase inhibitors include but are not limited to low molecular weight inhibitors, antibodies or antibody fragments, antisense constructs, small inhibitory RNAs (i.e. RNA interference by dsRNA; RNAi), and ribozymes. In a preferred embodiment, the EGFR kinase inhibitor is a small organic molecule or an antibody that binds specifically to the human EGFR.

EGFR kinase inhibitors that include, for example quinazoline EGFR kinase inhibitors, pyrido-pyrimidine EGFR kinase inhibitors, pyrimido-pyrimidine EGFR kinase inhibitors, pyrrolo-pyrimidine EGFR kinase inhibitors, pyrazolo-pyrimidine EGFR kinase inhibitors, phenylamino-pyrimidine EGFR kinase inhibitors, oxindole EGFR kinase inhibitors, indolocarbazole EGFR kinase inhibitors, phthalazine EGFR kinase inhibitors, isoflavone EGFR kinase inhibitors, quinalone EGFR kinase inhibitors, and tyrphostin EGFR kinase inhibitors, such as those described in the following patent publications, and all pharmaceutically acceptable salts and solvates of said EGFR kinase inhibitors: International Patent Publication Nos. WO 96/33980, WO 96/30347, WO 97/30034, WO 97/30044, WO 97/38994, WO 97/49688, WO 98/02434, WO 97/38983, WO 95/19774, WO 95/19970, WO 97/13771, WO 98/02437, WO 98/02438, WO 97/32881, WO 98/33798, WO 97/32880, WO 97/3288, WO 97/02266, WO 97/27199, WO 98/07726, WO 97/34895, WO 96/31510, WO 98/14449, WO 98/14450, WO 98/14451, WO 95/09847, WO 97/19065, WO 98/17662, WO 99/35146, WO 99/35132, WO 99/07701, and WO 92/20642; European Patent Application Nos. EP 520722, EP 566226, EP 787772, EP 837063, and EP 682027; U.S. Pat. Nos. 5,747,498, 5,789,427, 5,650,415, and 5,656,643; and German Patent Application No. DE 19629652. Additional non-limiting examples of low molecular weight EGFR kinase inhibitors include any of the EGFR kinase inhibitors described in Traxler, P., 1998, Exp. Opin. Ther. Patents 8(12):1599-1625.

Specific preferred examples of low molecular weight EGFR kinase inhibitors that can be used according to the present invention include [6,7-bis(2-methoxyethoxy)₄-quinazolin-4-yl]-(3-ethynylphenyl) amine (also known as OSI-774, erlotinib, or Tarceva™ (erlotinib HCl); OSI Pharmaceuticals/Genentech/Roche) (U.S. Pat. No. 5,747,498; International Patent Publication No. WO 01/34574, and Moyer, J. D. et al. (1997) Cancer Res. 57:4838-4848); CI-1033 (formerly known as PD183805; Pfizer) (Sherwood et al., 1999, Proc. Am. Assoc. Cancer Res. 40:723); PD-158780 (Pfizer); AG-1478 (University of California); CGP-59326 (Novartis); PKI-166 (Novartis); EKB-569 (Wyeth); GW-2016 (also known as GW-572016 or lapatinib ditosylate; GSK); and gefitinib (also known as ZD1839 or Iressa™; Astrazeneca) (Woodburn et al., 1997, Proc. Am. Assoc. Cancer Res. 38:633). A particularly preferred low molecular weight EGFR kinase inhibitor that can be used according to the present invention is [6,7-bis(2-methoxyethoxy)-4-quinazolin-4-yl]-(3-ethynylphenyl) amine (i.e. erlotinib), its hydrochloride salt (i.e. erlotinib HCl, Tarceva™), or other salt forms (e.g. erlotinib mesylate).

Antibody-based EGFR kinase inhibitors include any anti-EGFR antibody or antibody fragment that can partially or completely block EGFR activation by its natural ligand. Non-limiting examples of antibody-based EGFR kinase inhibitors include those described in Modjtahedi, H., et al., 1993, Br. J. Cancer 67:247-253; Teramoto, T., et al., 1996, Cancer 77:639-645; Goldstein et al., 1995, Clin. Cancer Res. 1:1311-1318; Huang, S. M., et al., 1999, Cancer Res. 15:59(8):1935-40; and Yang, X., et al., 1999, Cancer Res. 59:1236-1243. Thus, the EGFR kinase inhibitor can be the monoclonal antibody Mab E7.6.3 (Yang, X. D. et al. (1999) Cancer Res. 59:1236-43), or Mab C225 (ATCC Accession No. HB-8508), or an antibody or antibody fragment having the binding specificity thereof. Suitable monoclonal antibody EGFR kinase inhibitors include, but are not limited to, IMC-C225 (also known as cetuximab or Erbitux™; Imclone Systems), ABX-EGF (Abgenix), EMD 72000 (Merck KgaA, Darmstadt), RH3 (York Medical Bioscience Inc.), and MDX-447 (Medarex/Merck KgaA).

Additional antibody-based EGFR kinase inhibitors can be raised according to known methods by administering the appropriate antigen or epitope to a host animal selected, e.g., from pigs, cows, horses, rabbits, goats, sheep, and mice, among others. Various adjuvants known in the art can be used to enhance antibody production.

Although antibodies useful in practicing the invention can be polyclonal, monoclonal antibodies are preferred. Monoclonal antibodies against EGFR can be prepared and isolated using any technique that provides for the production of antibody molecules by continuous cell lines in culture. Techniques for production and isolation include but are not limited to the hybridoma technique originally described by Kohler and Milstein (Nature, 1975, 256: 495-497); the human B-cell hybridoma technique (Kosbor et al., 1983, Immunology Today 4:72; Cote et al., 1983, Proc. Natl. Acad. Sci. USA 80: 2026-2030); and the EBV-hybridoma technique (Cole et al, 1985, Monoclonal Antibodies and Cancer Therapy, Alan R. Liss, Inc., pp. 77-96).

Alternatively, techniques described for the production of single chain antibodies (see, e.g., U.S. Pat. No. 4,946,778) can be adapted to produce anti-EGFR single chain antibodies. Antibody-based EGFR kinase inhibitors useful in practicing the present invention also include anti-EGFR antibody fragments including but not limited to F(ab′).sub.2 fragments, which can be generated by pepsin digestion of an intact antibody molecule, and Fab fragments, which can be generated by reducing the disulfide bridges of the F(ab′).sub.2 fragments. Alternatively, Fab and/or scFv expression libraries can be constructed (see, e.g., Huse et al., 1989, Science 246: 1275-1281) to allow rapid identification of fragments having the desired specificity to EGFR.

Techniques for the production and isolation of monoclonal antibodies and antibody fragments are well-known in the art, and are described in Harlow and Lane, 1988, Antibodies: A Laboratory Manual, Cold Spring Harbor Laboratory, and in J. W. Goding, 1986, Monoclonal Antibodies: Principles and Practice, Academic Press, London. Humanized anti-EGFR antibodies and antibody fragments can also be prepared according to known techniques such as those described in Vaughn, T. J. et al., 1998, Nature Biotech. 16:535-539 and references cited therein, and such antibodies or fragments thereof are also useful in practicing the present invention.

EGFR kinase inhibitors for use in the present invention can alternatively be based on antisense oligonucleotide constructs. Anti-sense oligonucleotides, including anti-sense RNA molecules and anti-sense DNA molecules, would act to directly block the translation of EGFR mRNA by binding thereto and thus preventing protein translation or increasing mRNA degradation, thus decreasing the level of EGFR kinase protein, and thus activity, in a cell. For example, antisense oligonucleotides of at least about 15 bases and complementary to unique regions of the mRNA transcript sequence encoding EGFR can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as EGFR kinase inhibitors for use in the present invention. EGFR gene expression can be reduced by contacting the tumor, subject or cell with a small double stranded RNA (dsRNA), or a vector or construct causing the production of a small double stranded RNA, such that expression of EGFR is specifically inhibited (i.e. RNA interference or RNAi). Methods for selecting an appropriate dsRNA or dsRNA-encoding vector are well known in the art for genes whose sequence is known (e.g. see Tuschi, T., et al. (1999) Genes Dev. 13(24):3191-3197; Elbashir, S. M. et al. (2001) Nature 411:494-498; Hannon, G. J. (2002) Nature 418:244-251; McManus, M. T. and Sharp, P. A. (2002) Nature Reviews Genetics 3:737-747; Bremmelkamp, T. R. et al. (2002) Science 296:550-553; U.S. Pat. Nos. 6,573,099 and 6,506,559; and International Patent Publication Nos. WO 01/36646, WO 99/32619, and WO 01/68836).

Ribozymes can also function as EGFR kinase inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of EGFR mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as EGFR kinase inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

The invention also encompasses a pharmaceutical composition that is comprised of an EGFR kinase inhibitor and artesunate combination in combination with a pharmaceutically acceptable carrier.

Preferably the composition is comprised of a pharmaceutically acceptable carrier and a non-toxic therapeutically effective amount of an EGFR kinase inhibitor compound and artesunate combination (including pharmaceutically acceptable salts of each component thereof).

Moreover, within this preferred embodiment, the invention encompasses a pharmaceutical composition for the treatment of disease, the use of which results in the inhibition of growth of neoplastic cells, benign or malignant tumors, or metastases, comprising a pharmaceutically acceptable carrier and a non-toxic therapeutically effective amount of an EGFR kinase inhibitor compound and artesunate combination (including pharmaceutically acceptable salts of each component thereof).

The term “pharmaceutically acceptable salts” refers to salts prepared from pharmaceutically acceptable non-toxic bases or acids. When a compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (cupric and cuprous), ferric, ferrous, lithium, magnesium, manganese (manganic and manganous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylameine, trimethylamine, tripropylamine, tromethamine and the like.

When a compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The pharmaceutical compositions of the present invention comprise an EGFR kinase inhibitor compound and artesunate combination (including pharmaceutically acceptable salts of each component thereof) as active ingredient, a pharmaceutically acceptable carrier and optionally other therapeutic ingredients or adjuvants. Other therapeutic agents may include those cytotoxic, chemotherapeutic or anti-cancer agents, or agents which enhance the effects of such agents, as listed above. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by an EGFR kinase inhibitor compound and artesunate combination (including pharmaceutically acceptable salts of each component thereof) of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, an EGFR kinase inhibitor compound and artesunate combination (including pharmaceutically acceptable salts of each component thereof) may also be administered by controlled release means and/or delivery devices. The combination compositions may be prepared by any of the methods of pharmacy. In general, such methods include a step of bringing into association the active ingredients with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and an EGFR kinase inhibitor compound and artesunate combination (including pharmaceutically acceptable salts of each component thereof). An EGFR kinase inhibitor compound and artesunate combination (including pharmaceutically acceptable salts of each component thereof), can also be included in pharmaceutical compositions in combination with one or more other therapeutically active compounds. Other therapeutically active compounds may include those cytotoxic, chemotherapeutic or anti-cancer agents, or agents which enhance the effects of such agents, as listed above.

Thus in one embodiment of this invention, a pharmaceutical composition can comprise an EGFR kinase inhibitor compound and artesunate in combination with an anticancer agent, wherein said anti-cancer agent is a member selected from the group consisting of alkylating drugs, antimetabolites, microtubule inhibitors, podophyllotoxins, antibiotics, nitrosoureas, hormone therapies, kinase inhibitors, activators of tumor cell apoptosis, and antiangiogenic agents.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably contains from about 0.05 mg to about 5 g of the active ingredient.

For example, a formulation intended for the oral administration to humans may contain from about 0.5 mg to about 5 g of active agent, compounded with an appropriate and convenient amount of carrier material that may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical sue such as, for example, an aerosol, cream, ointment, lotion, dusting powder, or the like. Further, the compositions can be in a form suitable for use in transdermal devices. These formulations may be prepared, utilizing an EGFR kinase inhibitor compound and artesunate combination (including pharmaceutically acceptable salts of each component thereof) of this invention, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing an EGFR kinase inhibitor compound and artesunate combination (including pharmaceutically acceptable salts of each component thereof) may also be prepared in powder or liquid concentrate form.

Dosage levels for the compounds of the combination of this invention will be approximately as described herein, or as described in the art for these compounds. It is understood, however, that the specific dose level for any particular patient will depend upon a variety of factors including the age, body weight, general health, sex, diet, time of administration, route of administration, rate of excretion, drug combination and the severity of the particular disease undergoing therapy.

This invention will be better understood from the Experimental Details that follow. However, one skilled in the art will readily appreciate that the specific methods and results discussed are merely illustrative of the invention as described more fully in the claims which follow thereafter, and are not to be considered in any way limited thereto.

### Experimental Details

Introduction

While both OSI-774 and artesunate (ART) have been well described concerning their activity against tumor cells, nothing was known about the effectiveness of a combination treatment and the underlying molecular determinants of cellular response. In the present investigation, we have analyzed the small molecule EGFR tyrosine kinase inhibitor OSI-774 in combination with artesunate (ART) in a panel of 14 GBM cell lines, in order to identify molecular factors that may determine the cellular response to this combination treatment. Therefore, ART was tested in combination with OSI-774 in GBM cell lines transduced with expression vectors carrying either wild-type or deletion-mutant EGFR cDNAs as well as in a panel of GBM cell lines with varying degrees of inherent EGFR expression. Furthermore, the comparative genomic hybridization technique was used to identify genomic imbalances that correlated with the IC₅₀ values of GBM cell lines for ART and OSI-774. Finally, a profile of genomic aberrations has been identified by hierarchical cluster analysis that predicted sensitivity or resistance of GBM cell lines to the combination of ART and OSI-774.

Materials and Methods

Drugs

OSI-774 ([6,7-bis(2-methoxy-ethoxy)quinazoline-4-yl]-(3-ethylphenyl)amine HCl) was obtained from OSI Pharmaceuticals (Uniondale, N.Y., U.S.A.). ART was obtained from Saokim Ltd. (Hanoi, Vietnam).

Cell Lines

The establishment of the parental human GBM cell line U-87MG and its derivatives which overexpress exogenous wild-type epidermal growth factor receptor (U-87MG.WT-2N), tyrosine kinase-deficient EGFR (U-87MG.DK-2N), EGFR with a genomic deletion of exons 2 to 7 (U-87MG.ΔEGFR), or control expression vector (U-87MG.LUX), respectively, has been described elsewhere (Huang, H-J S et al. (1997) J. Biol. Chem. 272:2927-2935). The cell lines were provided by Dr. W. K. Cavenee (Ludwig Institute for Cancer Research, San Diego, Calif., U.S.A.). Cell culture conditions of these cell lines were as described (Nagane M, et al. (1996) Cancer Res. 56:5079-5086).

Nine established GBM cell lines derived from histopathologically confirmed neurosurgical specimens obtained in the Department for Neurosurgery, University of Gottingen, Germany, were maintained in RPMI 1640 medium (BioWhittaker, Walkersville, Md., U.S.A.) supplemented with 10% heat-inactivated fetal calf serum and incubated in a humidified 5% CO₂ atmosphere at 37° C. Medium was exchanged twice weekly, and cells were passaged upon reaching subconfluence. At the beginning of the study, all cell lines were beyond their 20^{th} passage.

Growth Inhibition Assay

The in vitro response to cytostatic drugs was evaluated by means of a growth inhibition assay. Aliquots of 5×10³ cells/ml were seeded in culture medium, and drugs were added at different concentrations. Cells were counted 10 days after seeding. These growth curves represent the net outcome of cell proliferation and cell death. Cell numbers were quantitated each in eight independent determinations. To detect additive or synergistic effects in combination treatments, OSI-774 and ART were applied at 50% isodoses and plotted in isobolograms as previously described (Steel, G. G. and Peckham, M. J. (1979) Int. J. Radiat. Oncol. Biol. Phys. 5:85-91; Berenbaum, M. C. (1981) Adv. Cancer Res. 35:269-335).

Colony Forming Assay

Tumor cells (2×10³ cells/ml) were seeded in 60-mm dishes. After changing the medium two days later, treatment with different drug concentrations was initiated. One week later, the colonies that grew from the surviving cells were fixed in ethanol acetic acid (3:1), stained with 0.1% toluidine blue, and counted.

Comparative Genomic Hybridization

DNA was extracted using TriStar™ reagent (Hybaid, Heidelberg, Germany) and subjected to comparative genomic hybridization (CGH) on metaphase chromosomes of normal peripheral lymphocytes prepared on slides. The CGH protocol used has been previously described in detail (Wolff E, et al. (1997) Int. J. Oncol. 11:19-23). The validity of this method has been proven on a series of leukemia cases (Gebhart, E. et al. (2000) Int. J. Oncol. 16:1099-1105; Verdorfer, I. et al. (2001) Cancer Genet. Cytogenet. 124:1-6). Briefly, 500 ng tumor DNA and 500 ng normal DNA were labeled with biotin and digoxigenin, respectively, by nick translation (Roche Diagnostics, Mannheim, Germany). The probe mixture of 500 ng tumor DNA, 500 ng normal DNA, 20 μg Cot-1 DNA (GIBCO BRL, Life Technologies, Eggenstein-Leopoldshafen, Germany) and 10 μg herring testis DNA (Sigma-Aldrich, Deisenhofen, Germany) was denatured in 50% formamide/20% dextran sulphate/2×SSC/50 mM sodium phosphate buffer for 3 min at 75° C. and preannealed at 37° C. for 20 min. The probe mixture was hybridized to normal lymphocyte metaphase slides denatured in 70% formamide/2×SSC/sodium phosphate buffer, and sealed with cover slides and rubber cement. The slides were incubated at 37° C. for three days in a humidified chamber. For fluorescence detection, the slides were stained with avidin-fluorescein isothiocyanate (Vector Laboratories, Burlingame, Calif., U.S.A.) and anti-digoxigenin-rhodamine (Roche Diagnostics, Mannheim, Germany), followed by counter-staining with 4′,6-diamidino-2-phenylindole dihydrochloride (DAPI). The slides were mounted in 20 μl anti-fade solution (Vectashields, Vector Labs) and evaluated using a fluorescence microscope (Axioplan Zeiss, Jena, Germany) equipped with a CCD camera (IMAC S30) and the ISIS 3 software (MetaSystems, Altlussheim, Germany). Gains or losses of genetic material were deemed as significant by the evaluation software if fluorescence ratio borderline values of 0.8 and 1.2 were not reached or exceeded, respectively. Twenty karyotypes were analyzed per experiment.

Statistical Analyses

Hierarchical cluster analysis is an explorative statistical method that groups seemingly heterogeneous objects into clusters of homogeneous objects. Objects are classified by calculation of distances according to the closeness of interindividual distances. All objects are assembled into a cluster tree (dendrogram). The merging of objects with similar features leads to the formation of a cluster, where the length of the branch indicates the degree of relation. The procedure continues to aggregate clusters until there is only one. The distance of subordinate clusters to a superior cluster represents a criterion for the closeness of clusters as well as for the affiliation of single objects to clusters. Thus, objects with tightly related features appear together, while the distance in the cluster tree increases with progressive dissimilarity. The applicability of cluster tree models for comparative genomic hybridization data has been demonstrated previously (Desper R, et al. (1999) J. Comput. Biol. 6:37-51; Efferth, T. et al. (2002) Blood Cells Mol. Dis. 29:1-13). Cluster analyses applying the complete-linkage method were done by means of the WinSTAT program (Kalmia, Cambridge, Mass., U.S.A.). Missing values were automatically omitted by the program, and the closeness of two joined objects was calculated by the number of data points they contained. In order to calculate distances of all variables included in the analysis, the program automatically standardizes the variables by transforming the data with mean=0 and variance=1. Standard statistical tests (Wilcoxon test, χ² test) were used as implements of the WinSTAT program.

Results and Discussion

Results

Growth Inhibition Assays

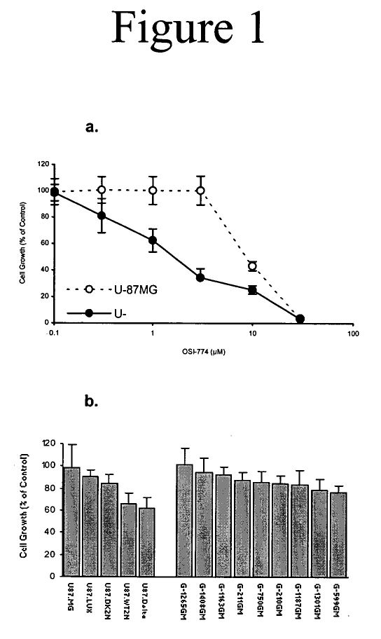

As a starting point, growth inhibition after exposure to OSI-774 has been determined in all glioblastoma cell lines investigated. OSI-774 has been applied in a concentration range from 0.1 to 30 μM to U-87MG.ΔEGFR cells transduced with a deletion-mutant EGFR and U-87MG control cells (FIG. 1a). The IC₅₀ values for OSI-774 calculated from the dose-response curves were 1.6 μM for U-87MG.ΔEGFR and 8.6 μM for U-87MG cells. Hence, U-87MG.ΔEGFR cells were 5.5-fold more sensitive to OSI-774 than U-87MG cells. These dose-response curves demonstrate that a single concentration of 1 μM OSI-774 is sufficient to differentiate between both cell lines. Further experiments with all other cell lines were, therefore, performed with this concentration of OSI-774 (1 μM). As shown in FIG. 1b, 1 μM OSI-774 inhibited growth of U-87MG.ΔEGFR by 38.28% and of U-87MG.WT-2N cells by 33.76%, whereas growth of U-87MG.DK-2N, U-87MG.LUX, and U-87MG cells was reduced by 15.5% to 1.72%. The inhibition of the non-transduced cell line panel by 1 μM OSI-774 ranged from 23.4% (G-599GM) to −1.79% (G-1265GM) (FIG. 1b).

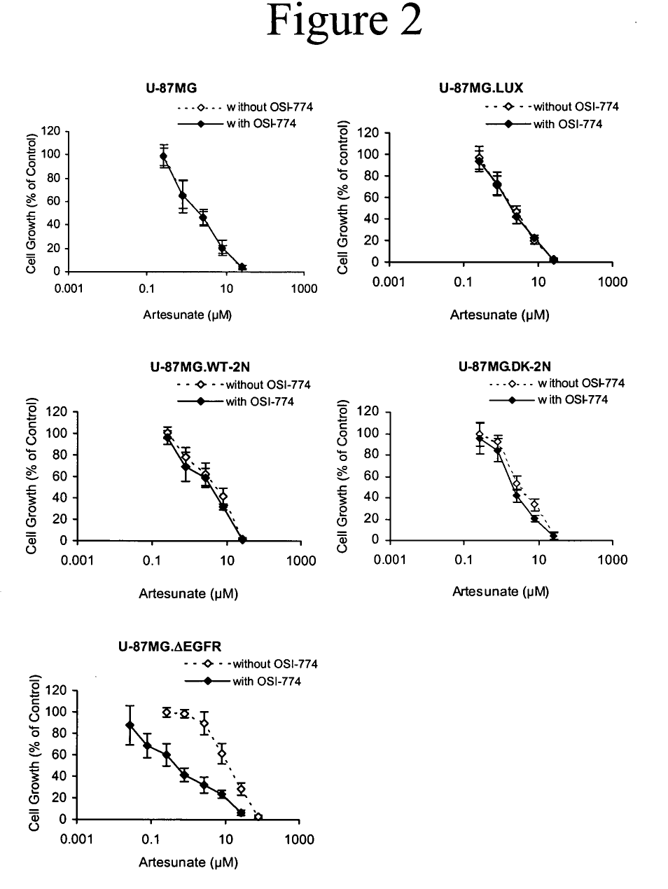

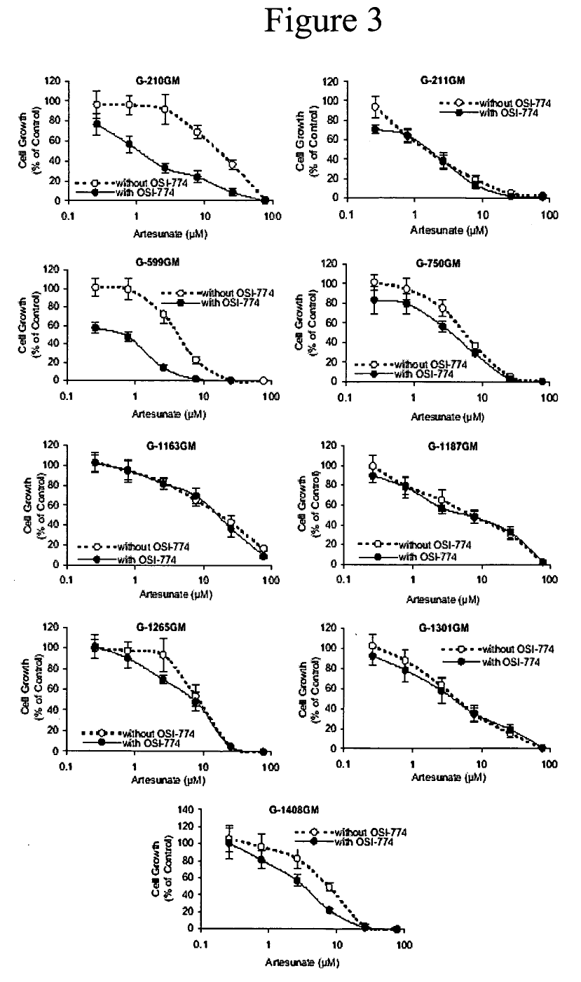

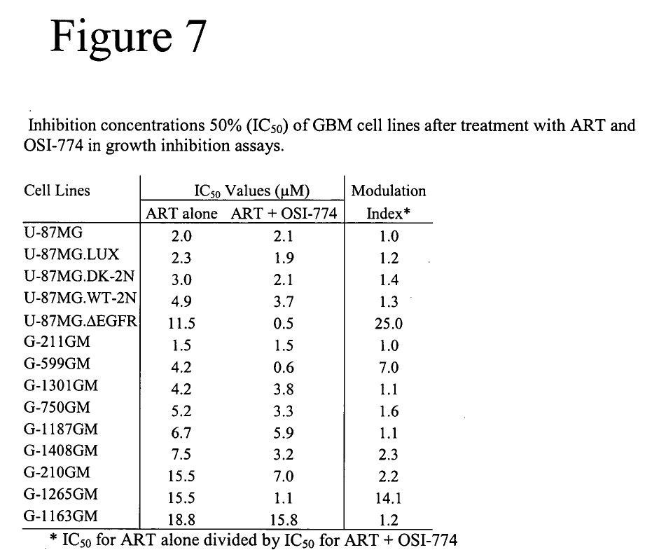

A concentration of 1 μM OSI-774 was also used for the combination treatment with ART, which was applied in a concentration range of 0.01 to 30 μg/ml (0.026 to 78 μM). As depicted in FIGS. 2 and 3, the ART-induced cell growth inhibition expressed as percentage of cell growth of untreated control cells varied considerably between the cell lines. The values of growth inhibition for 1 μM OSI-774 alone were set as 100%, and cell growth rates under the combination treatment of ART and OSI-774 were related to these control values. This means that a strictly additive effect of both drugs appears as an overlapping dose-response curve compared to treatment with ART alone, while supra-additive effects of ART plus OSI-774 are visible as a curve below that for ART alone. As depicted in FIG. 2, the combination of ART plus OSI-774 led to a strong increase of growth inhibition compared to ART alone in U-87MG.ΔEGFR cells but not in U-87MG.WT-2N, U-87MG.DK-2N, U-87MG.LUX, or U-87MG cells. The degree of increase of inhibition by ART plus OSI-774 compared to ART alone was 25-fold in U-87MG.ΔEGFR cells, while it ranged from 0.95 to 1.4-fold in the other U-87MG cell lines (FIG. 7).

Among 9 non-transduced GBM cell lines, the G-210GM and G-599GM cell lines showed increased growth inhibitions by the combination of both drugs with multiplicities of 14.09 and 6.77-fold, respectively, compared to treatment with ART alone (FIGS. 3 and 7), indicating supra-additive effects. ART plus OSI-774 caused modestly increased growth inhibitions compared to ART alone in G-750GM and G-1408GM cell lines (1.58 and 2.34-fold, respectively). The other five cell lines displayed no or only minimal increases of growth inhibition (<1.5-fold), indicating that ART plus OSI-774 act strictly additive in these cell lines. Sub-additive or antagonistic effects were not observed at all.

Isobolographic Analyses

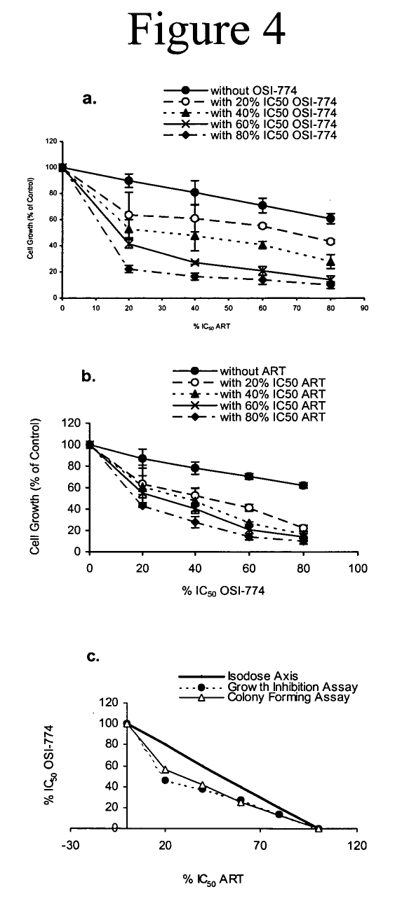

These observations were then confirmed using isobolographic analyses. We used the G-599GM cell line for these experiments, because it showed a supra-additive modulation of ART by OSI-774 compared to ART alone in growth inhibition assays. In the first step, OSI-774 alone was applied in different concentrations. The IC₅₀ value calculated from this dose-response curve was 1.3 μM. The IC₅₀ value for ART alone was 4.2 μM in G-599GM cells (FIG. 7). In a second step, both drugs were then applied together in concentrations of 20%, 40%, 60%, and 80% of the corresponding IC₅₀ values of each drug alone. FIG. 4a shows that the dose-dependent inhibition of ART alone was enhanced by addition of OSI-774. The increase in growth inhibition rose with serially increasing OSI-774 concentrations. Plotting of the data against OSI-774 at the x-axis (FIG. 4b) indicated that the increasing IC₅₀ fractions of ART enhanced the growth inhibitory activity of OSI-774. Plotting of the combination treatment IC₅₀ values of OSI-774 against ART revealed a supra-additive inhibition of cell growth (FIG. 4c). The results obtained by means of the growth inhibition assay were then scrutinized using a second independent test method. As shown in FIG. 4c, comparable results were found by the colony-forming assay.

Comparative Genomic Hybridization and Hierarchical Cluster Analysis

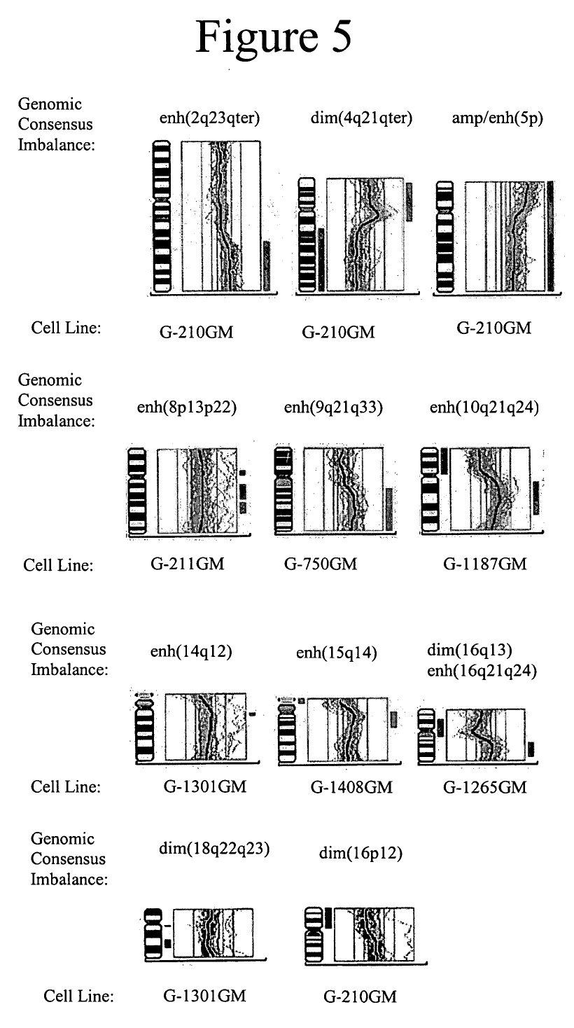

The genomic imbalances (gains or losses of genetic material) in the non-transduced GBM cell lines have been described by our group (Ramirez, T. et al. (2003) Int. J. Oncol. 23:453-60). In the present investigation, all genomic aberrations detected in these cell lines were correlated with the IC₅₀ values for ART and OSI-774 by means of the Wilcoxon test. Those imbalances which showed a relationship to cellular drug response at P<0.09 were subjected to further analyses. Representative chromosomal profiles of genomic hybridizations harboring these imbalances are shown in FIG. 5. Enhancements or amplifications of genetic material are marked by a bar to the right and losses by a bar to the left in each chromosomal graph.

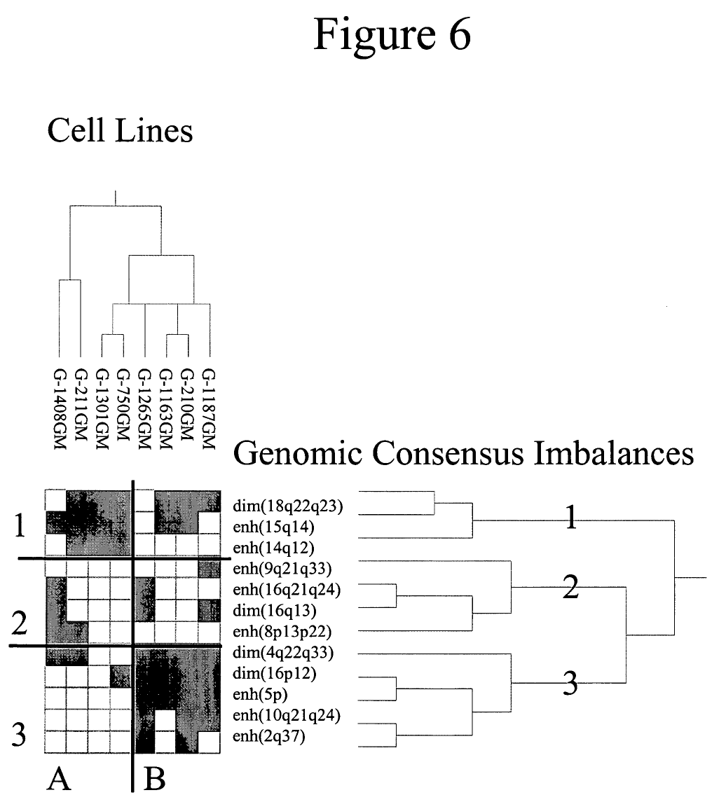

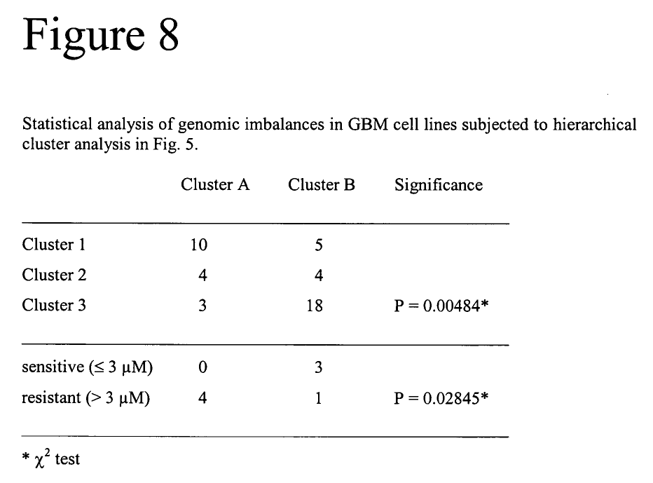

These genomic imbalances of the GBM cell lines were then investigated by hierarchical cluster analysis and clustered image mapping in order to detect genomic profiles of imbalances that predict sensitivity or resistance to ART and OSI-774. The upper dendrogram in FIG. 6 could be divided into two major clusters (clusters A and B), and the dendrogram on the right into three major clusters (clusters 1, 2, and 3). First, we examined whether the distribution of genomic imbalances was different between the clusters with statistical significance. As shown in FIG. 8, genomic imbalances of clusters 1 and 3 of the dendrogram on the right (FIG. 6) were differentially distributed between clusters A and B of the upper dendrogram (P=0.00484; X² test), while genomic imbalances of cluster 2 were not. Then, we correlated the two clusters on the top of FIG. 6 with the IC₅₀ values for OSI-774 and ART that were not included into the cluster analysis beforehand (FIG. 8). The distribution of sensitive and resistant GBM cell lines was significantly different among these two clusters (P=0.02845). At a cut-off IC₅₀ value of 3 μM, cluster B contained cell lines that were more sensitive to ART and OSI-774, while cluster A was enriched with more resistant cell lines. As can be seen in the cluster image map in FIG. 6, the genomic imbalances of cluster 1 (dim(18q22q23), enh(15q14), and enh(14q12)) were more frequently present in the “resistant” cluster A, whereas the genomic aberrations of cluster 3 (dim(4q22q33), dim(16p12), enh(5p), enh(10q21q24), and enh(2q37)) were more frequently present in the “sensitive” cluster B.

Discussion

Additive and Supra-Additive Growth Inhibition by ART and OSI-774

In the present investigation, we analyzed the combination treatment of ART and OSI-774 in a panel of 14 GBM cell lines. OSI-774 was applied in a concentration of 1 μM in combination with ART. In a recent phase I study on patients with advanced solid malignancies (Hildalgo, M. et al. (2001) J. Clin. Oncol. 19:3267-79), the average minimal steady state plasma concentration of OSI-774 was 1.2 μg/ml (3 μM). Hence, the concentration of 1 μM that was active in the present investigation is safely within the range of clinically achievable plasma concentrations. The same applies to ART. The IC₅₀ values for ART in 14 GBM cell lines ranged from 1.5 to 18.5 μM. OSI-774 modulated ART-induced growth inhibition in these cell lines by reducing the IC₅₀ range from 0.6 to 15.8 μM. In clinical anti-malarial studies, peak plasma concentrations of 2640±1800 μg/ml (6.88±4.69 μM) have been achieved upon intravenous application of 2 mg/kg ART (Batty, K. T. J. et al. (1996) Chromatogr. Biomed. Appl. 677:345-50). Hence, peak plasma concentrations were in a comparable range as the IC₅₀ values for ART in the present study. As the doses of both ART and OSI-774 were well within clinically relevant ranges, the results obtained in this investigation might gain relevance for the inhibition of tumor cell growth in humans.

An EGFR gene deletion mutation comprising exons 2 to 7 conferred resistance to ART, whereas the wild-type EGFR gene or EGFR with deficient tyrosine kinase activity did not. Unlike wild-type EGFR, the gene product of deletion-mutant EGFR is constitutively active in a ligand-independent manner (Huang, H-J S et al. (1997) J. Biol. Chem. 272:2927-2935), indicating that the extent of stimulated EGFR autophosphorylation influences cellular response to ART. This is also known for other anticancer drugs like cisplatin, doxorubicin, homoharringtonine, paclitaxel, or vincristine (Dickstein B. M. (1995) Mol. Cell. Endocrinol. 110:205-11; Nagane, M. et al. (1998) Proc. Natl. Acad. Sci. USA 95:5724-5729; Efferth T. (2003) Arch. Pharmacol. 367:56-67). As EGFR affects resistance to these and multiple other structurally and functionally unrelated drugs, it can be assumed that EGFR-mediated drug resistance may be yet another variant of pleiotropic drug resistance in addition to the classical multidrug resistance phenotypes caused by ATP-binding cassette transporter genes, i.e., MDR1, MRP, or BCRP.

In contrast to treatment with ART alone, we observed that U-87MG.ΔEGFR cells were even more sensitive to treatment with OSI-774 alone than the control cell lines U-87MG and U-87MG.LUX. This may be explained by a strong dependency of ΔEGFR-expressing cells on a permanently high activity of EGFR-downstream signaling pathways. Upon disruption of EGFR pathway signaling by OSI-774 the U-87MG.ΔEGFR cells were more inhibited than the other U-87MG cell lines. This observation fits to recent results showing that OSI-774 acts preferentially on cells with highly malignant phenotypes (Halatsch, M-E. et al. (2000) J. Neurosurg. 92:297-305).

Recent studies have demonstrated that EGFR may regulate cellular responses to cytotoxic drugs by regulation of key molecules of apoptosis, e.g., BCL2, BCL-X, MYC, and NFκB1 (Catz S. D. and Johnson J. L. (2001) Oncogene 20:7342-51; Glasgow, J. N. (2001) Neurochem. Res. 26:647-59; Arcinas, M. et al. (2001) Cancer Res. 61:5202-6) either by activation of transcription factors, i.e., CREB and FOS, or by phosphorylation of target proteins. Roles of EGFR, RAF, AKT, PKC, the BCL2 gene family, NFκB1, and FOS for resistance to established anti-tumor drugs have been demonstrated previously (Boldogh, I. et al. (1998) Cancer Res. 58:3950-3956; Tergaonkar, V. et al. (2002) Cancer Cell. 1:493-503; Davis, J. M. et al. (2003) Clin. Cancer Res. 9:1161-1170; Navolanic, P. M. et al. (2003) Int. J. Oncol. 22:237-252), suggesting that downstream pathways of EGFR signaling may also contribute to the response of tumor cells to ART.

The fact that the overexpression of constitutively activated EGFR decreases sensitivity of GBM to ART renders inhibitors of EGFR attractive candidates for combination treatments with this compound. In vitro and in vivo studies have demonstrated additive to synergistic effects of small molecule tyrosine kinase inhibitors or monoclonal antibodies against EGFR in combination with established anticancer drugs (Efferth, T. and Volm, M. (1992) Med. Oncol. Tumor Pharmacother. 9:11-9; Raben, D, et al. (2002) Semin. Oncol. 29:(Suppl. 4):3746). This has also been observed for antibodies against a closely related gene of EGFR, HER2, and cytostatic drugs (Toma, S. et al. (2002) J. Cell Physiol. 193:37-41). Comparable results have been found for OSI-774 in combination with established anticancer drugs (Pollack, V. A. et al. (1999) J Pharmacol. Exp. Ther. 291:739-748). In the present investigation with a panel of GBM cell lines, we found that OSI-774 plus ART produced additive and supra-additive inhibition in cell growth and colony forming assays. These results support the hypothesis of Mendelsohn and Fan (Mendelsohn, J. and Fan, D. (1997) J. Natl. Cancer Inst. 89:341-343) that chemotherapy converts EFGR ligands from growth factors to survival factors. Repression of EGFR protein function by specific inhibitors in combination with cytotoxic drugs may cause irreversible cell damage that leads to apoptosis.

Comparative Genomic Hybridization and Hierarchical Cluster Analysis

All of the non-transduced cell lines examined contained an enhancement of the short arm of chromosome 7 where the EGFR gene is located and expressed or overexpressed EGFR mRNA and protein to a different extent (Halatsch, M-E et al. (2003) Anticancer Res. 23:2315-2320; Ramirez, T. et al. (2003) Int. J. Oncol. 23:453-60). Although OSI-774 inhibits downstream EGFR signaling in a specific manner, the response of our panel of GBM cell lines to ART and OSI-774 is, however, not strictly determined by EGFR expression, and other factors may also contribute to the tumor cells' responses to these drugs.

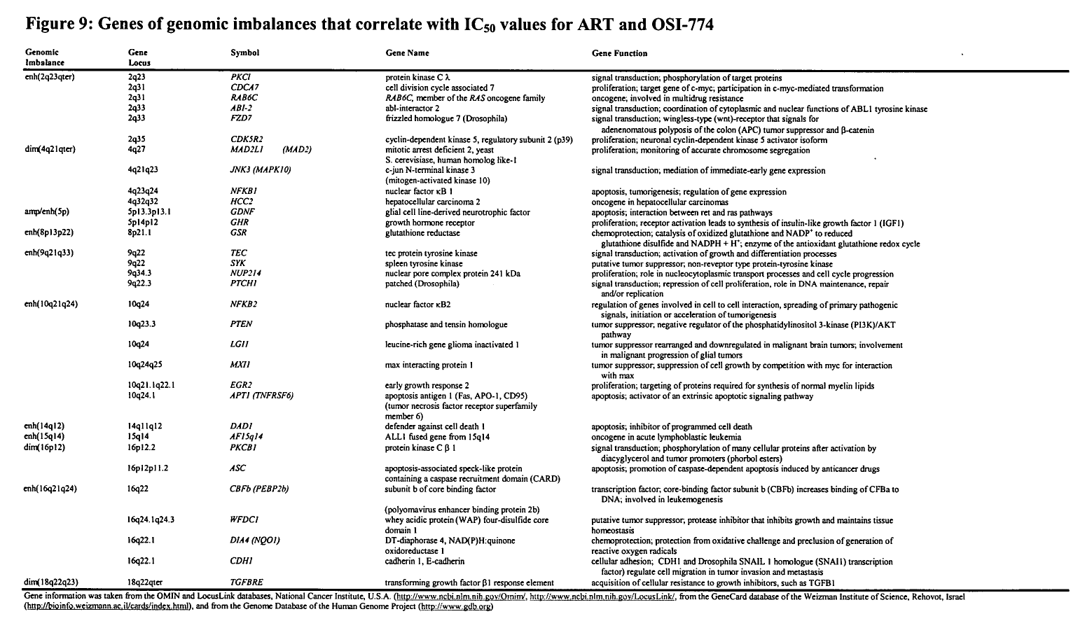

The comparative genomic hybridization technique was used to detect all genetic imbalances of each cell line at the genomic level in one single hybridization experiment. This approach allows to correlate the presence of genomic aberrations with the individual IC₅₀ values for ART and OSI-774 in these cell lines and thereby to identify genomic regions that are associated with drug sensitivity and resistance. The genes located at these genomic loci (FIG. 9) may influence the action of ART and OSI-774 in the individual cell lines. The genes belong to classes of different biological functions, e.g., genes involved in apoptosis, proliferation, signal transduction, or xenobiotic detoxification as well as oncogenes and tumor suppressor genes. Some of these genes have been already shown previously to cause resistance to established anticancer drugs, i.e., GSR, PKCB], NFKB1, TNFRSF6 (CD95), and RAB6C (Fan, D. et al. (1992) Anticancer Res.12:661-667; Hao, X. Y. et al. (1994) Carcinogenesis 15:1167-1173; Friesen, C. et al. (1997) Leukemia 11:1833-1841; Shan, J. et al. (2000) Gene 257:67-75; Tergaonkar, V. et al. (2002) Cancer Cell. 1:493-503), some of them being targets of EGFR signaling pathways as discussed above. By means of hierarchical cluster analysis we have shown here that it is indeed possible to predict sensitivity or resistance of glioblastoma cell lines to ART and OSI-774 based on their genomic imbalances. Consequently, certain genes allocated to the respective genomic loci may contribute to drug sensitivity and resistance. In how far the genes identified by our approach are causally related to sensitivity or resistance to ART and OSI-774 requires further investigation.

In summary, we have demonstrated that the combination of ART and OSI-774 provoked additive to supra-additive growth inhibition in 14 GBM cell lines. These effects were correlated either with deletion-mutation-driven constitutive activation of EGFR in transduced cell lines or with several genomic imbalances in non-transduced cell lines. Out results warrant further studies to dissect the molecular architecture that determines sensitivity and resistance of tumor cells to OSI-774 and ART in more detail.

### Incorporation by Reference

All patents, published patent applications, and other references disclosed herein are hereby expressly incorporated herein by reference.

### Equivalents

Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A pharmaceutical composition comprising an EGFR kinase inhibitor and artesunate in a pharmaceutically acceptable carrier.

2. The pharmaceutical composition of claim 1, wherein the EGFR kinase inhibitor comprises erlotinib.

3. The pharmaceutical composition of claim 1, additionally comprising one or more additional anti-cancer agents.

4. A composition in accordance with claim 3, wherein said additional anti-cancer agent is a member selected from alkylating drugs, antimetabolites, microtubule inhibitors, podophyllotoxins, antibiotics, nitrosoureas, hormone therapies, kinase inhibitors, activators of tumor cell apoptosis, and antiangiogenic agents.

5. A method for treating tumors or tumor metastases in a patient, comprising administering to said patient simultaneously or sequentially a therapeutically effective amount of an EGFR kinase inhibitor and artesunate.

6. The method of claim 5, wherein the patient is a human that is being treated for cancer.

7. The method of claim 5, wherein the EGFR kinase inhibitor and artesunate are co-administered to the patient in the same formulation.

8. The method of claim 5, wherein the EGFR kinase inhibitor and artesunate are co-administered to the patient in different formulations.

9. The method of claim 5, wherein the EGFR kinase inhibitor and artesunate are co-administered to the patient by the same route.

10. The method of claim 5, wherein the EGFR kinase inhibitor and artesunate are co-administered to the patient by different routes.

11. The method of claim 5, wherein the EGFR kinase inhibitor is administered to the patient by parenteral or oral administration.

12. The method of claim 5, wherein artesunate is administered to the patient by parenteral administration.

13. The method of claim 5, wherein the tumors or tumor metastases to be treated are selected from lung cancer, colorectal cancer, NSCLC, bronchioloalviolar cell lung cancer, bone cancer, pancreatic cancer, skin cancer, cancer of the head or neck, cutaneous melanoma, intraocular melanoma, uterine cancer, ovarian cancer, rectal cancer, anal region cancer, stomach cancer, gastric cancer, colon cancer, breast cancer, uterine cancer, fallopian tube carcinoma, endometrial carcinoma, cervical carcinoma, vaginal carcinoma, vulval carcinoma, Hodgkin's Disease, esophagus cancer, small intestine cancer, endocrine system cancer, thyroid gland cancer, parathyroid gland cancer, adrenal gland cancer, soft tissue sarcoma, urethral cancer, penis cancer, prostate cancer, bladder cancer, kidney cancer, ureter cancer, renal cell carcinoma, renal pelvis carcinoma, mesothelioma, hepatocellular cancer, biliary cancer, chronic leukemia, acute leukemia, lymphocytic lymphoma, CNS neoplasm, spinal axis cancer, glioma, brain stem glioma, glioblastoma multiforme, astrocytoma, schwannoma, ependymoma, medulloblastoma, meningioma, squamous cell carcinoma and pituitary adenoma tumors or tumor metastases.

14. The method of claim 13, wherein the tumors or tumor metastases are refractory.

15. The method of claim 13, wherein the tumors or tumor metastases to be treated are glioma tumors or tumor metastases.

16. The method of claim 5, wherein the EGFR kinase inhibitor comprises erlotinib.

17. The method of claim 5, additionally comprising administering one or more other anti-cancer agents.

18. The method of claim 17, wherein the other anti-cancer agents are selected from an alkylating agent, cyclophosphamide, chlorambucil, cisplatin, carboplatin, oxaliplatin, busulfan, melphalan, carmustine, streptozotocin, triethylenemelamine, mitomycin C, an anti-metabolite, methotrexate, etoposide, 6-mercaptopurine, 6-thiocguanine, cytarabine, 5-fluorouracil, capecitabine, gemcitabine, dacarbazine, an antibiotic, actinomycin D, doxorubicin, daunorubicin, bleomycin, mithramycin, an alkaloid, vinblastine, paclitaxel, docetaxel, vinorelbine, a glucocorticoid, dexamethasone, a corticosteroid, prednisone, a nucleoside enzyme inhibitors, hydroxyurea, an amino acid depleting enzyme, asparaginase, topotecan, irinotecan, leucovorin, and a folic acid derivative.

19. A method for the treatment of cancer, comprising administering to a subject in need of such treatment a first amount of an EGFR kinase inhibitor, or a pharmaceutically acceptable salt thereof; and a second amount of artesunate; wherein at least one of the amounts is administered as a sub-therapeutic amount.

20. The method of claim 19, wherein the cancer is a glioma.
